(19) United States
(12) Patent Application Publication
(10) Pub. No.: US 2018/0062089 A1
(43) Pub. Date: Mar. 1, 2018

(54) COMPOUND, COMPOSITION AND ORGANIC LIGHT-EMITTING DEVICE

(71) Applicants: Cambridge Display Technology Limited, Godmanchester (GB); Sumitomo Chemical Company Limited, Tokyo (JP)

(72) Inventor: Kiran KAMTEKAR, Godmanchester (GB)

(73) Assignees: Cambridge Display Technology Limited, Godmanchester (GB); Sumitomo Chemical Company Limited, Tokyo (JP)

(21) Appl. No.: 15/551,399

(22) PCT Filed: Feb. 16, 2016
(86) PCT No.: PCT/GB2016/050377

§ 371 Date: Aug. 16, 2017

(30) Foreign Application Priority Data
Feb. 16, 2015 (GB) 1502587.7

(51) Int. Cl. H01L 51/00; C07F 7/08; H01L 51/50; C07D 209/86

(52) U.S. Cl. CPC H01L 51/0094; C07F 7/0812; C07F 7/0814; C07D 209/86; H01L 51/0054; H01L 51/50; H01L 51/0073

(57) ABSTRACT

Compound, Composition and Organic Light-Emitting Device A compound of formula (I) (Formula (I)) wherein X is O, S, NR⁸, CR⁸₂ or SiR⁸₂ wherein R⁸ in each occurrence is independently a substituent; R¹, R⁵ and R⁶ are independently in each occurrence a substituent; x independently in each occurrence is 0, 1, 2, 3 or 4; and y independently in each occurrence is 0, 1 or 2. The compound may be provided as a sidechain, end group or backbone group of a polymer. The compound may be used as a host for a phosphorescent light-emitting material in an organic light-emitting device.

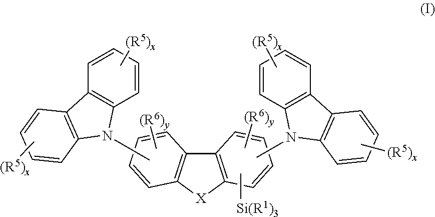

### BACKGROUND OF THE INVENTION

Electronic devices containing active organic materials are attracting increasing attention for use in devices such as organic light emitting diodes (OLEDs), organic photoresponsive devices (in particular organic photovoltaic devices and organic photosensors), organic transistors and memory array devices. Devices containing active organic materials offer benefits such as low weight, low power consumption and flexibility. Moreover, use of soluble organic materials allows use of solution processing in device manufacture, for example inkjet printing or spin-coating.

An OLED may comprise a substrate carrying an anode, a cathode and one or more organic light-emitting layers between the anode and cathode.

Holes are injected into the device through the anode and electrons are injected through the cathode during operation of the device. Holes in the highest occupied molecular orbital (HOMO) and electrons in the lowest unoccupied molecular orbital (LUMO) of a light-emitting material combine to form an exciton that releases its energy as light.

Light-emitting materials include small molecule, polymeric and dendrimeric materials. Light-emitting polymers include poly(arylene vinylenes) such as poly(p-phenylene vinylenes) and polymers containing arylene repeat units, such as fluorene repeat units.

A light emitting layer may comprise a host material and a light-emitting dopant wherein energy is transferred from the host material to the light-emitting dopant. For example, J. Appl. Phys. 65, 3610, 1989 discloses a host material doped with a fluorescent light-emitting dopant (that is, a light-emitting material in which light is emitted via decay of a singlet exciton).

Phosphorescent dopants are also known (that is, a light-emitting dopant in which light is emitted via decay of a triplet exciton).

Sook et al, J. Mater. Chem., 2011, 21, 14604 discloses host materials DBT1, DBT2 and DBT3:

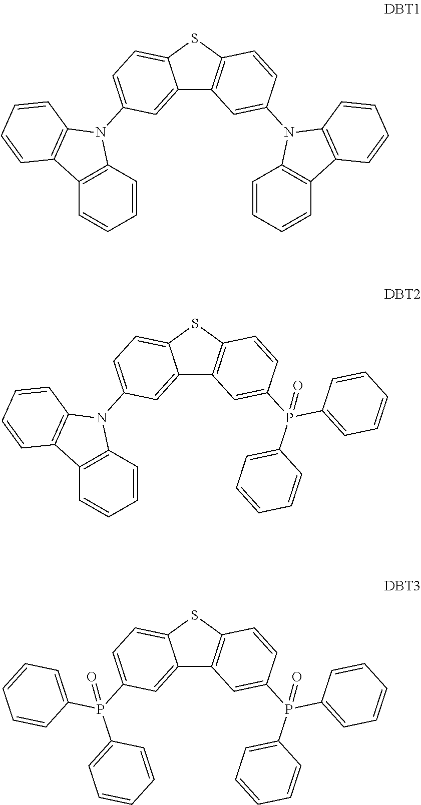 [SMILES: c1ccc2c(c1)c1ccccc1n2-c1ccc2sc3ccc(-n4c5ccccc5c5ccccc54)cc3c2c1 ; O=P(c1ccccc1)(c1ccccc1)c1ccc2sc3ccc(-n4c5ccccc5c5ccccc54)cc3c2c1 ; O=P(c1ccccc1)(c1ccccc1)c1ccc2sc3ccc(P(=O)(c4ccccc4)c4ccccc4)cc3c2c1]

US 2013/293094 discloses hosts containing an electron-transport moiety and a hole-transport moiety separated by an aromatic spacer and a triarylsilane group.

U.S. Pat. No. 6,562,982 discloses hosts having the following formula wherein Ar is an aryl and R¹, R², R³ and R⁴ are independently a hydrocarbyl:

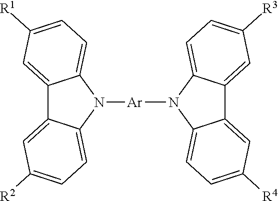

It is an object of the invention to provide a host for light-emitting dopants, in particular phosphorescent light-emitting dopants, for high efficiency OLEDs.

It is a further object of the invention to provide a host for blue phosphorescent light-emitting dopants.

### SUMMARY OF THE INVENTION

In a first aspect the invention provides a compound of formula (I)

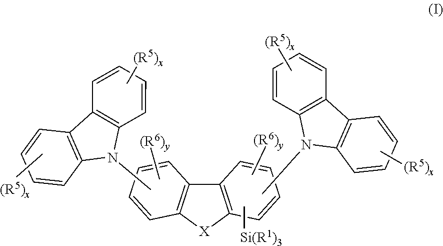

wherein:

X is O, S, NR⁸, CR⁸₂ or SiR⁸₂ wherein R⁸ in each occurrence is independently a substituent;

R¹, R⁵ and R⁶ are independently in each occurrence a substituent;

x independently in each occurrence is 0, 1, 2, 3 or 4; and

y independently in each occurrence is 0, 1 or 2.

In a second aspect the invention provides a composition comprising a compound according to the first aspect and at least one light-emitting dopant.

In a third aspect the invention provides a formulation comprising a compound according to the first aspect or a composition according to the second aspect and at least one solvent.

In a fourth aspect the invention provides an organic light-emitting device comprising an anode, a cathode and one or more organic layers between the anode and cathode including a light-emitting layer wherein at least one of the one or more organic layers comprises a compound according to the first aspect.

In a fifth aspect the invention provides a method of forming an organic light-emitting device according to the fourth aspect, the method comprising the step of forming the light-emitting layer over one of the anode and the cathode and forming the other of the anode and the cathode over the light-emitting layer.

In a sixth aspect the invention provides a polymer having a polymer backbone and comprising a group of formula (II) in the polymer backbone, as a side-chain of the polymer backbone or and end group of the polymer backbone:

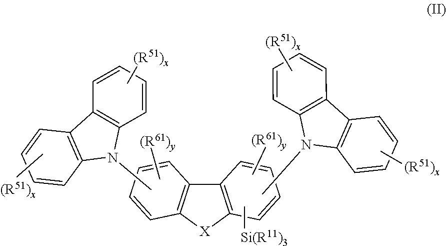

wherein X is O, S, NR⁸, CR⁸₂ or SiR⁸₂ wherein R⁸ in each occurrence is independently a substituent;

R¹¹, R⁵¹ and R⁶¹ are independently in each occurrence a substituent;

x independently in each occurrence is 0, 1, 2, 3 or 4; and

y independently in each occurrence is 0, 1 or 2,

with the proviso that at least one of R⁵¹, R⁶¹ and R¹¹ is bound to the polymer backbone.

The polymer of the sixth aspect may be used as described herein with reference to compounds of formula (I).

The polymer of the sixth aspect may be provided in a composition comprising the polymer and at least one light-emitting dopant.

The polymer of the sixth aspect may be provided in a formulation comprising the polymer and at least one solvent.

The polymer of the sixth aspect may be provided in an organic light-emitting device comprising an anode, a cathode and one or more organic layers between the anode and cathode including a light-emitting layer wherein at least one of the one or more organic layers comprises the polymer. The device may be as described anywhere herein. The device may be formed by a method comprising the step of forming the light-emitting layer over one of the anode and the cathode and forming the other of the anode and the cathode over the light-emitting layer.

In a seventh aspect the invention provides a compound of formula (V):

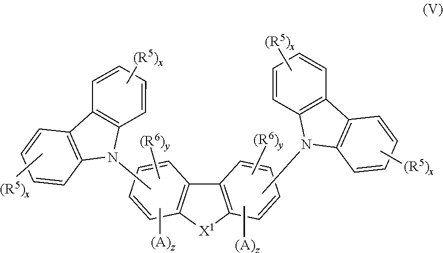

wherein:

X¹ is O or S;

each A is a LUMO-deepening substituent;

R⁵ and R⁶ are independently in each occurrence a substituent;

x independently in each occurrence is 0, 1, 2, 3 or 4;

y independently in each occurrence is 0, 1 or 2, and

each z is independently 0 or 1 with the proviso that at least one z is 1.

R⁵ and R⁶, x and y of the seventh aspect may be as described anywhere herein with reference to compounds of formula (I). Compounds of the seventh aspect may be used as described herein with reference to compounds of formula (I).

Compounds of formula (V) may be provided in the polymer backbone, as a side-chain of a polymer backbone or and end group of the polymer backbone.

By “LUMO deepening substituent” is meant a substituent that results in a compound of formula (V) having a LUMO level that is deeper (further from vacuum) than a corresponding compound in which the substituent A is replaced with H. Optionally, A is a group of formula —Si(R¹)₃.

### DESCRIPTION OF THE DRAWINGS

The invention will now be described in more detail with reference to the drawings in which:

FIG. 1 illustrates an OLED according to an embodiment of the invention.

### DETAILED DESCRIPTION OF THE INVENTION

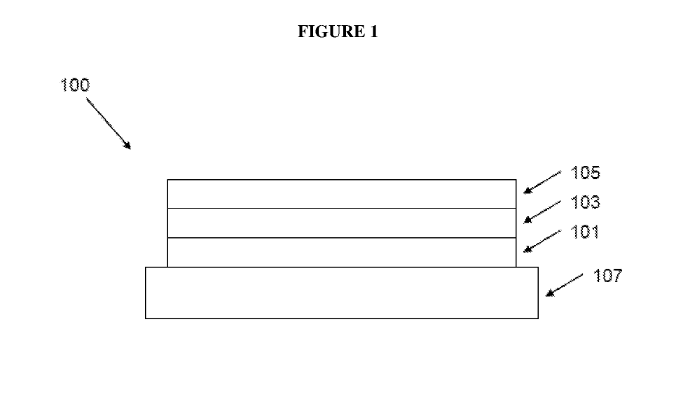

FIG. 1 illustrates an OLED 100 according to an embodiment of the invention comprising an anode 101, a cathode 105 and a light-emitting layer 103 between the anode and cathode. The device 100 is supported on a substrate 107, for example a glass or plastic substrate.

One or more further layers may be provided between the anode 101 and cathode 105, for example hole-transporting layers, electron transporting layers, hole blocking layers and electron blocking layers. The device may contain more than one light-emitting layer.

Preferred device structures include:

Anode/Hole-injection layer/Light-emitting layer/Cathode

Anode/Hole transporting layer/Light-emitting layer/Cathode

Anode/Hole-injection layer/Hole-transporting layer/Light-emitting layer/Cathode

Anode/Hole-injection layer/Hole-transporting layer/Light-emitting layer/Electron-transporting layer/Cathode.

Preferably, at least one of a hole-transporting layer and hole injection layer is present.

Preferably, both a hole injection layer and hole-transporting layer are present.

Light-emitting materials include red, green and blue light-emitting materials.

A blue emitting material may have a photoluminescent spectrum with a peak in the range of 400-490 nm, optionally 420-490 nm.

A green emitting material may have a photoluminescent spectrum with a peak in the range of more than 490 nm up to 580 nm, optionally more than 490 nm up to 540 nm

A red emitting material may optionally have a peak in its photoluminescent spectrum of more than 580 nm up to 630 nm, optionally 585-625 nm.

The photoluminescent spectrum of a light-emitting material may be measured from its phosphorescence spectrum by casting 5 wt % of the material in a PMMA film onto a quartz substrate to achieve transmittance values of 0.3-0.4 and measuring in a nitrogen environment using apparatus C9920-02 supplied by Hamamatsu.

Light-emitting layer 103 may contain a compound of formula (I) doped with one or more luminescent dopants. The light-emitting layer 103 may consist essentially of these materials or may contain one or more further materials, for example one or more charge-transporting materials, optionally a hole-transporting material, and/or one or more further light-emitting materials. When used as a host material for one or more light-emitting dopants, the lowest excited stated singlet (S¹) or the lowest excited state triplet (T¹) energy level of the host material is preferably no more than 0.1 eV below that of a fluorescent or phosphorescent light-emitting material respectively, and is more preferably about the same as or higher than that of the light-emitting material in order to avoid quenching of luminescence from the light-emitting dopant.

In the case where the luminescent dopant is a phosphorescent dopant, the compound of formula (I) preferably has a T¹ of greater than 2.8 eV, preferably greater than 3.0 eV.

Triplet energy levels of compounds of formula (I) may be measured from the energy onset of the phosphorescence spectrum measured by low temperature phosphorescence spectroscopy (Y. V. Romaovskii et al, Physical Review Letters, 2000, 85 (5), p 1027, A. van Dijken et al, Journal of the American Chemical Society, 2004, 126, p 7718).

The triplet energy level of a light-emitting material may be measured from its phosphorescence spectrum by casting 5 wt % of the material in a PMMA film onto a quartz substrate to achieve transmittance values of 0.3-0.4 and measuring in a nitrogen environment using apparatus C9920-02 supplied by Hamamatsu.

In a preferred embodiment, light-emitting layer 103 contains a compound of formula (I) and at least one of green and blue phosphorescent light-emitting materials.

The compound of formula (I) may have formula (Ia):

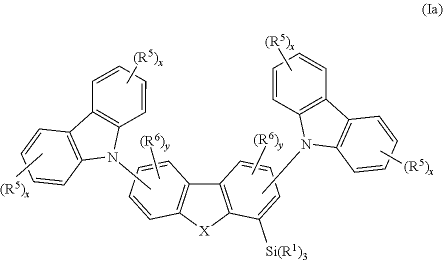

Each R¹ of formula (I) may independently be selected from the group consisting of:

alkyl, optionally C₁₋₂₀ alkyl, wherein one or more non-adjacent C atoms may be replaced with optionally substituted aryl or heteroaryl, O, S, substituted N, C═O or —COO—, and one or more H atoms may be replaced with F; and

(Ar¹)ₚ wherein Ar¹ independently in each occurrence is a C₆₋₂₀ aryl or heteroaryl group that may be unsubstituted or substituted with one or more substituents, and p is at least 1, optionally 1, 2 or 3.

Preferably, R¹ is (Ar¹)ₚ. Exemplary Ar¹ groups are phenyl, fluorene, dibenzothiophene, dibenzofuran and carbazole.

Each Ar¹ group may be unsubstituted or substituted with one or more substituents. Optionally, substituents for Ar¹ are selected from C₁₋₂₀ alkyl wherein one or more non-adjacent C atoms may be replaced O, S, C═O or —COO—, and one or more H atoms may be replaced with F.

Exemplary groups R¹ are illustrated below:

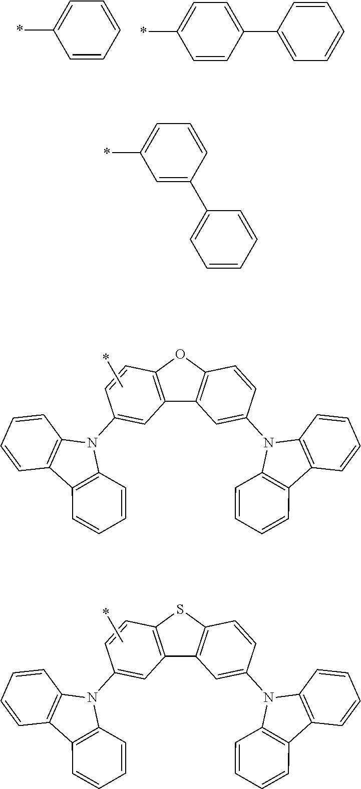

wherein * represents a bond to Si, and where each aryl or heteroaryl group may be unsubstituted or substituted with one or more substituents.

Each R⁵ and R⁶, where present, may independently in each occurrence be selected from the group consisting of alkyl, optionally C₁₋₂₀ alkyl, wherein one or more non-adjacent C atoms may be replaced with optionally substituted aryl or heteroaryl, O, S, substituted N, C═O or —COO—, and one or more H atoms may be replaced with F; aryl and heteroaryl groups that may be unsubstituted or substituted with one or more substituents, preferably phenyl substituted with one or more C₁₋₂₀ alkyl groups; F; CN and NO₂.

Preferably, each x is 0.

Preferably, each y is 0.

Where present, R⁸ is preferably a C₁₋₄₀ hydrocarbyl group, optionally a C₁₋₂₀ alkyl group or phenyl that may be unsubstituted or substituted with one or more C₁₋₂₀ alkyl groups.

Preferably, X is S or O.

Preferably, the carbazole groups of formula (I) or (Ia) are bound to the 2- and 8-positions of a dibenzothiopehene (X═S) or dibenzofuran (X═O) group of formula (I).

Exemplary compounds of formula (I) include the following:

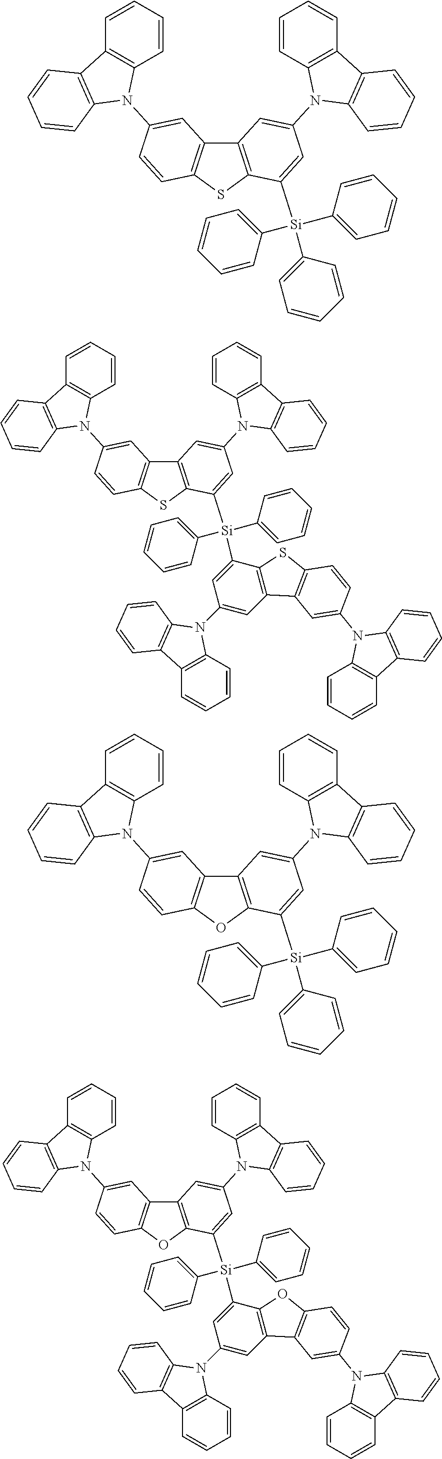

The compound of formula (I) may be provided in a polymer having a polymer backbone and comprising a group of formula (II) in the polymer backbone, as a side-chain of the polymer backbone or and end group of the polymer backbone:

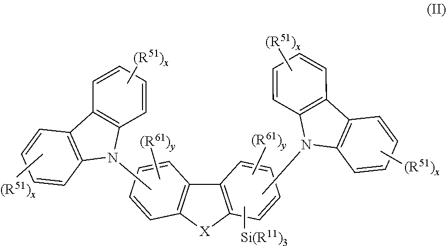

wherein X is O, S, NR⁸, CR⁸₂ or SiR⁸₂ wherein R⁸ in each occurrence is independently a substituent;

R¹¹, R⁵¹ and R⁶¹ are independently in each occurrence a substituent;

x independently in each occurrence is 0, 1, 2, 3 or 4; and

y independently in each occurrence is 0, 1 or 2,

with the proviso that at least one of R⁵¹, R⁶¹ and R¹¹ is bound to the polymer backbone.

It will be understood that the polymer may be used as described herein with respect to compounds of formula (I).

R¹¹, R⁵¹ and R⁶¹ may each independently be as described with respect to R¹, R⁵ and R⁶ respectively with the proviso that at least one of R⁵¹, R⁶¹ and R¹¹ is bound to the polymer backbone.

If only one of R⁵¹, R⁶¹ and R¹¹ is bound to the polymer backbone then the group of formula (II) may be an end-group of the polymer or a side-group of a repeat unit of the polymer.

If two or more, preferably two, of R⁵¹, R⁶¹ and R¹¹ are bound to the polymer backbone then the group of formula (II) may be a repeat unit in the backbone of the polymer.

Optionally, the group of formula (II) is a repeat unit of formula (IIa) or (IIb):

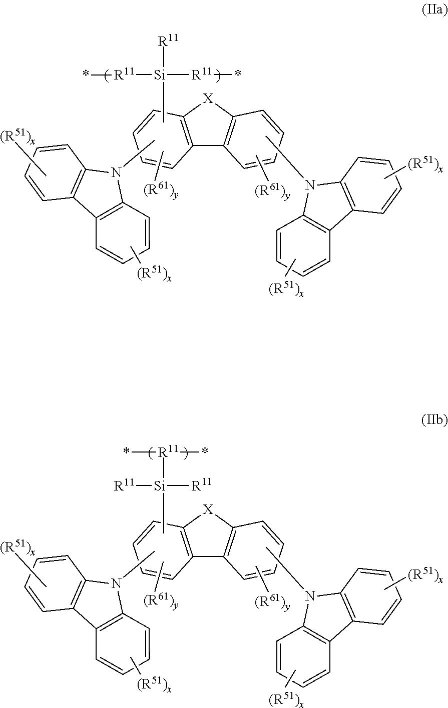

The repeat units of formulae (IIa) and (IIb) may be formed by polymerisation of monomers of formulae (IIIa) and (IIIb) respectively:

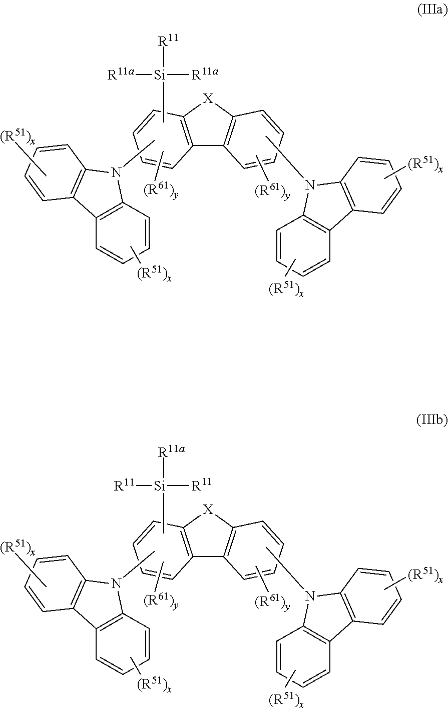

wherein R^{11a} is a polymerisable group.

Optionally, each R^{11a} of the compound of formula (IIIa) is independently an aryl or heteroaryl group, optionally phenyl, that is substituted with a leaving group. Suitable leaving groups are halogen, preferably bromine or iodine, boronic acid and boronic esters. Monomers of formula (IIIa) may be polymerized by Suzuki polymerisation, for example as described in WO 00/53656, or Yamamoto polymerisation.

Optionally, R11a of the compound of formula (IIIb) is selected from the group consisting of: an aryl or heteroaryl group, optionally phenyl, that is substituted with leaving groups; and a group comprising a terminal alkene unit.

A group R11a comprising a terminal alkene unit may have formula (IV):

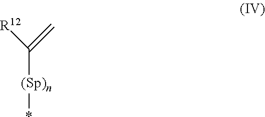

wherein R¹² is H or a substituent, optionally H or a C₁₋₂₀ alkyl group; Sp is a spacer group; n is 0 or 1; and * is a point of attachment to Si.

Optionally, Sp is selected from (i) an ester group of formula —C(═O)O—R¹⁴— wherein R¹⁴ is a direct bond or a C₁₋₁₀ alkyl group, and (ii) a C₁₋₂₀ hydrocarbyl group, optionally a group selected from alkyl; phenyl; phenylalkyl; and alkylphenyl. The group of formula (IV) may comprise an acrylate group or alkylacrylate, optionally a methacrylate, group.

Suitable leaving groups are halogen, preferably bromine or iodine, boronic acid and boronic esters. Monomers of formula (IIIb) comprising leaving groups may be polymerized by Suzuki polymerisation, for example as described in WO 00/53656, or Yamamoto polymerisation.

A monomer comprising a terminal alkene unit may be polymerized, either alone or in combination with one or more further monomers, to form a polymer having a non-conjugated backbone.

A monomer of formula (IIIa) or (IIIb) comprising leaving groups may be polymerized, either alone or in combination with one or more further monomers, to form a polymer having a conjugated backbone. Optionally, the further monomers comprise monomers for forming C₆₋₂₀ arylene co-repeat units, optionally repeat units selected from phenylene, fluorene and phenanthrene co-repeat units.

A polymer comprising a group of formula (II) as described herein may be mixed with one or more light-emitting compounds as described herein, or the polymer may be covalently bound to a light-emitting compound.

A compound of formula (I) or (V) as described herein, or a polymer comprising repeating units comprising a unit of formula (I) or (V), preferably has a LUMO level that is more than 2.00 eV from vacuum level, optionally at least 2.10 eV or at least 2.20 eV from vacuum level, wherein the LUMO level is measured by square wave voltammetry as described herein.

Light-Emitting Compounds

A preferred use of compounds of formula (I) is as the host material for a light-emitting material in a light-emitting layer of an OLED.

Suitable light-emitting materials for a light-emitting layer include polymeric, small molecule and dendritic light-emitting materials, each of which may be fluorescent or phosphorescent.

A light-emitting layer of an OLED may be unpatterned, or may be patterned to form discrete pixels. Each pixel may be further divided into subpixels. The light-emitting layer may contain a single light-emitting material, for example for a monochrome display or other monochrome device, or may contain materials emitting different colours, in particular red, green and blue light-emitting materials for a full-colour display.

A light-emitting layer may contain more than one light-emitting material, for example a mixture of light-emitting materials that together provide white light emission.

A white-emitting OLED may contain a single, white-emitting layer containing a light-emitting composition as described herein, or may contain two or more layers that emit different colours which, in combination, produce white light and wherein at least one of the light emitting layers contains a composition as described herein. Optionally, a white-emitting OLED comprises red, green and blue light-emitting materials.

The light emitted from a white-emitting OLED may have CIE x coordinate equivalent to that emitted by a black body at a temperature in the range of 2500-9000K and a CIE y coordinate within 0.05 or 0.025 of the CIE y co-ordinate of said light emitted by a black body, optionally a CIE x coordinate equivalent to that emitted by a black body at a temperature in the range of 2700-6000K.

Exemplary phosphorescent light-emitting compounds have formula (IX):

ML¹_{q}L²ᵣL³ₛ (IX)

wherein M is a metal; each of L¹, L² and L³ is a coordinating group that independently may be unsubstituted or substituted with one or more substituents; q is a positive integer; r and s are each independently 0 or a positive integer; and the sum of (a·q)+(b·r)+(c·s) is equal to the number of coordination sites available on M, wherein a is the number of coordination sites on L¹, b is the number of coordination sites on L² and c is the number of coordination sites on L³.

a, b and c are preferably each independently 1, 2 or 3. Preferably, a, b and c are each a bidentate ligand (a, b and c are each 2). In an embodiment, q is 3 and r and s are 0. In another embodiment, q is 1 or 2, r is 1 and s is 0 or 1.

Heavy elements M induce strong spin-orbit coupling to allow rapid intersystem crossing and emission from triplet or higher states. Suitable heavy metals M include d-block metals, in particular those in rows 2 and 3 i.e. elements 39 to 48 and 72 to 80, in particular ruthenium, rhodium, palladium, rhenium, osmium, iridium, platinum and gold. Iridium is particularly preferred.

Exemplary ligands L¹, L² and L³ include carbon or nitrogen donors such as porphyrin or bidentate ligands of formula (X):

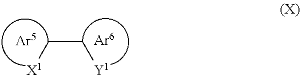

wherein Ar⁵ and Ar⁶ may be the same or different and are independently selected from substituted or unsubstituted aryl or heteroaryl; X¹ and Y¹ may be the same or different and are independently selected from carbon or nitrogen; and Ar⁵ and Ar⁶ may be fused together. Ligands wherein X¹ is carbon and Y¹ is nitrogen are preferred, in particular ligands in which Ar⁵ is a single ring or fused heteroaromatic of N and C atoms only, for example pyridyl or isoquinoline, and Ar⁶ is a single ring or fused aromatic, for example phenyl or naphthyl.

To achieve red emission, Ar⁵ may be selected from phenyl, fluorene, naphthyl and Ar⁶ are selected from quinoline, isoquinoline, thiophene and benzothiophene.

To achieve green emission, Ar⁵ may be selected from phenyl or fluorene and Ar⁶ may be pyridine.

To achieve blue emission, Ar⁵ may be selected from phenyl and Ar⁶ may be selected from imidazole, pyrazole, triazole and tetrazole.

Examples of bidentate ligands are illustrated below:

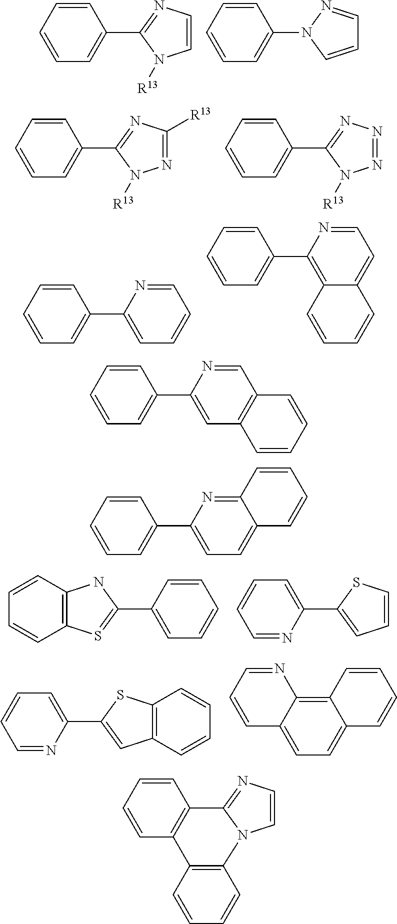

wherein R¹³ is a substituent.

Each of Ar⁵ and Ar⁶ may carry one or more substituents. Two or more of these substituents may be linked to form a ring, for example an aromatic ring.

Other ligands suitable for use with d-block elements include N,N-bidentate ligands, optionally bipyridyl; N,O-bidentate ligands, optionally picolinate; and O,O-bidentate ligands, optionally diketonates, in particular acetylacetonate (acac), tetrakis-(pyrazol-1-yl)borate, 2-carboxypyridyl, triarylphosphines and pyridine, each of which may be substituted.

One or more of L¹, L² and L³ may comprise a carbene group. Optionally, L¹ has formula:

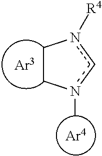

wherein R⁴ is H or a substituent and Ar³ and Ar⁴ are each independently a monocyclic or fused aryl or heteroaryl group.

Optionally, the phosphorescent compound has formula (XII):

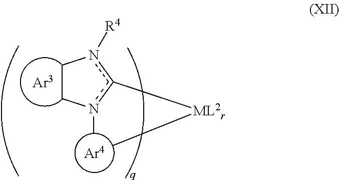

wherein M is a second or third row transition metal;

q is at least 1, optionally 1, 2 or 3;

r is 0 or a positive integer;

R⁴ is H or a substituent;

Ar³ and Ar⁴ are each independently a monocyclic or fused aryl or heteroaryl group; and

L² is a ligand that does not comprise a carbene group.

Optionally, R⁴ is selected from the group consisting of C₁₋₂₀ alkyl and a group of formula —(Ar⁵)ₜ wherein Ar⁵ is a C₆₋₂₀ aryl group or 5-20 membered heteroaryl group and t is at least 1, optionally 1, 2 or 3.

Ar⁵ is preferably phenyl.

Each Ar⁵ may independently be unsubstituted or substituted with one or more substituents, optionally one or more C₁₋₂₀ alkyl groups.

Ar³ is preferably a monocyclic heteroaromatic group of C and N atoms, optionally pyridine or pyrazine.

Ar⁴ is preferably a C₆₋₂₀ aryl group, more preferably phenyl.

Ar³ and Ar⁴ may each independently be unsubstituted or substituted with one or more substituents R² wherein each R² is independently selected from the group consisting of:

D;

F;

CN;

C₁₋₂₀ alkyl, wherein one or more non-adjacent C atoms may be replaced with optionally substituted aryl or heteroaryl (preferably phenyl), O, S, C═O or —COO—, and one or more H atoms may be replaced with F; and

—(Ar⁵)ₜ as described above.

Exemplary ligands L² are N,N-bidentate ligands, optionally bipyridyl; N,O-bidentate ligands, optionally picolinate; and O,O-bidentate ligands, optionally acac.

Optionally, the group of formula (XII) has formula (XIIa):

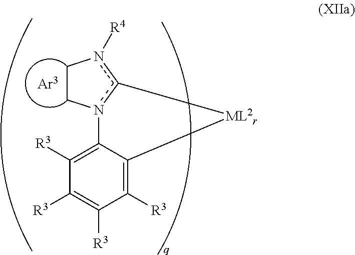

wherein each R³ is independently H or a substituent R² as described above.

Optionally, the group of formula (XII) has formula (XIIb):

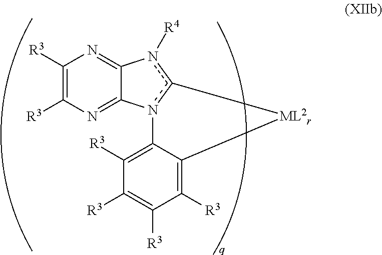

Preferably, compositions described herein comprise a compound of formula (I) and a blue phosphorescent material, more preferably a blue phosphorescent material of formula (IX) wherein L¹, independently in each occurrence if q is more than 1, is selected from a carbene or a ligand of formula (X), optionally phenylimidazole; phenyltriazole or unsubstituted or substituted phenylpyridine; and L² and L³, if present, are independently selected from N,N-, N,O and O,O-bidentate ligands. Exemplary substituents include groups R¹³ as described below with reference to Formula (VII). Particularly preferred substituents include fluorine or trifluoromethyl which may be used to blue-shift the emission of the complex, for example as disclosed in WO 02/45466, WO 02/44189, US 2002-117662 and US 2002-182441; alkyl or alkoxy groups, for example C₁₋₂₀ alkyl or alkoxy, which may be as disclosed in JP 2002-324679; carbazole which may be used to assist hole transport to the complex when used as an emissive material, for example as disclosed in WO 02/81448; phenyl or biphenyl which may be unsubstituted or substituted with one or more C₁₋₁₀ alkyl groups; and dendrons which may be used to obtain or enhance solution processability of the metal complex, for example as disclosed in WO 02/66552.

A light-emitting dendrimer comprises a light-emitting core bound to one or more dendrons, wherein each dendron comprises a branching point and two or more dendritic branches. Preferably, the dendron is at least partially conjugated, and at least one of the branching points and dendritic branches comprises an aryl or heteroaryl group, for example a phenyl group. In one arrangement, the branching point group and the branching groups are all phenyl, and each phenyl may independently be substituted with one or more substituents, for example alkyl or alkoxy.

A dendron may have optionally substituted formula (XI)

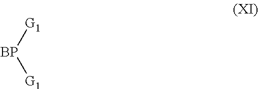

wherein BP represents a branching point for attachment to a core and G₁ represents first generation branching groups.

The dendron may be a first, second, third or higher generation dendron. G₁ may be substituted with two or more second generation branching groups G₂, and so on, as in optionally substituted formula (XIa):

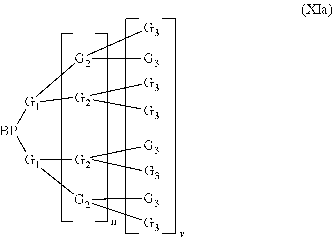

wherein u is 0 or 1; v is 0 if u is 0 or may be 0 or 1 if u is 1; BP represents a branching point for attachment to a core and G₁, G₂ and G₃ represent first, second and third generation dendron branching groups. In one preferred embodiment, each of BP and G₁, G₂ . . . Gₙ is phenyl, and each phenyl BP, G₁, G₂ . . . Gₙ₋₁ is a 3,5-linked phenyl.

A preferred dendron is a substituted or unsubstituted dendron of formula (XIb):

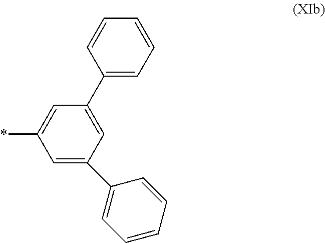 [SMILES: *c1cc(-c2ccccc2)cc(-c2ccccc2)c1]

wherein * represents an attachment point of the dendron to a core.

BP and/or any group G may be substituted with one or more substituents, for example one or more C₁₋₂₀ alkyl or alkoxy groups.

Light-emitting material(s) in a composition comprising or consisting of the compound of formula (I) and one or more light-emitting materials may make up about 0.05 wt % up to about 50 wt %, optionally about 1-40 wt % of the composition.

Charge Transporting and Charge Blocking Layers

A device containing a light-emitting layer containing a compound of formula (I) may have charge-transporting and/or charge blocking layers.

A hole transporting layer may be provided between the anode and the light-emitting layer or layers of an OLED. An electron transporting layer may be provided between the cathode and the light-emitting layer or layers.

An electron blocking layer may be provided between the anode and the light-emitting layer(s) and a hole blocking layer may be provided between the cathode and the light-emitting layer(s). Charge-transporting and charge-blocking layers may be used in combination. Depending on the HOMO and LUMO levels of the material or materials in a layer, a single layer may both transport one of holes and electrons and block the other of holes and electrons.

If present, a hole transporting layer located between the anode and the light-emitting layer(s) preferably has a material having a HOMO level of less than or equal to 5.5 eV, more preferably around 4.8-5.5 eV or 4.9-5.3 eV as measured by cyclic voltammetry. The HOMO level of the material in the hole transport layer may be selected so as to be within 0.2 eV, optionally within 0.1 eV of the light-emitting material of the light-emitting layer.

A hole-transporting layer may contain polymeric or non-polymeric charge-transporting materials. Exemplary hole-transporting materials contain arylamine groups.

A hole transporting layer may contain a homopolymer or copolymer comprising a repeat unit of formula (VII):

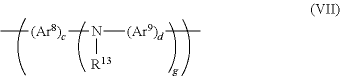

wherein Ar⁸ and Ar⁹ in each occurrence are independently selected from substituted or unsubstituted aryl or heteroaryl, g is greater than or equal to 1, preferably 1 or 2, R¹³ is H or a substituent, preferably a substituent, and c and d are each independently 1, 2 or 3.

R¹³, which may be the same or different in each occurrence when g>1, is preferably selected from the group consisting of alkyl, for example C₁₋₂₀ alkyl, Ar¹⁰, a branched or linear chain of Ar¹⁰ groups, or a crosslinkable unit that is bound directly to the N atom of formula (VIII) or spaced apart therefrom by a spacer group, wherein Ar¹⁰ in each occurrence is independently optionally substituted aryl or heteroaryl. Exemplary spacer groups are C₁₋₂₀ alkyl, phenyl and phenyl-C₁₋₂₀ alkyl.

Any of Ar⁸, Ar⁹ and, if present, Ar¹⁰ in the repeat unit of Formula (VII) may be linked by a direct bond or a divalent linking atom or group to another of Ar⁸, Ar⁹ and Ar¹⁰. Preferred divalent linking atoms and groups include O, S; substituted N; and substituted C.

Any of Ar⁸, Ar⁹ and, if present, Ar¹⁰ may be substituted with one or more substituents. Exemplary substituents are substituents R¹⁰, wherein each R¹⁰ may independently be selected from the group consisting of:
substituted or unsubstituted alkyl, optionally C₁₋₂₀ alkyl, wherein one or more non-adjacent C atoms may be replaced with optionally substituted aryl or heteroaryl, O, S, substituted N, C═O or —COO— and one or more H atoms may be replaced with F; and
a crosslinkable group attached directly to Ar⁸, Ar⁹ or Ar¹⁰ or spaced apart therefrom by a spacer group, for example a group comprising a double bond such and a vinyl or acrylate group, or a benzocyclobutane group

Preferred repeat units of formula (VII) have formulae 1-3:

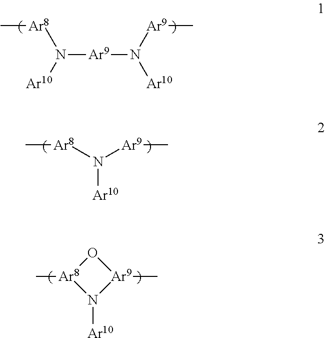

In one preferred arrangement, R¹³ is Ar¹⁰ and each of Ar⁸, Ar⁹ and Ar¹⁰ are independently and optionally substituted with one or more C₁₋₂₀ alkyl groups. Ar⁸, Ar⁹ and Ar¹⁰ are preferably phenyl.

In another preferred arrangement, the central Ar⁹ group of formula (1) linked to two N atoms is a polycyclic aromatic that may be unsubstituted or substituted with one or more substituents R¹⁰. Exemplary polycyclic aromatic groups are naphthalene, perylene, anthracene and fluorene.

In another preferred arrangement, Ar⁸ and Ar⁹ are phenyl, each of which may be substituted with one or more C₁₋₂₀ alkyl groups, and R¹³ is —(Ar¹⁰)ᵣ wherein r is at least 2 and wherein the group —(Ar¹⁰)ᵣ forms a linear or branched chain of aromatic or heteroaromatic groups, for example 3,5-diphenylbenzene wherein each phenyl may be substituted with one or more C₁₋₂₀ alkyl groups. In another preferred arrangement, c, d and g are each 1 and Ar⁸ and Ar⁹ are phenyl linked by an oxygen atom to form a phenoxazine ring.

A hole-transporting polymer containing repeat units of formula (VII) may be a copolymer containing one or more further repeat units. Exemplary further repeat units include arylene repeat units, each of which may be unsubstituted or substituted with one or more substituents.

Exemplary arylene repeat units include without limitation, fluorene, phenylene, naphthalene, anthracene, indenofluorene, phenanthrene and dihydrophenanthrene repeat units, each of which may be unsubstituted or substituted with one or more substituents.

Substituents of arylene repeat units, if present, may be selected from C₁₋₄₀ hydrocarbyl, preferably C₁₋₂₀ alkyl; phenyl which may be unsubstituted or substituted with one or more C₁₋₁₀ alkyl groups; and crosslinkable hydrocarbyl groups, for example C₁₋₄₀ hydrocarbyl groups comprising benzocyclobutene or vinylene groups.

Phenylene repeat units may be 1,4-linked phenylene repeat units that may be unsubstituted or substituted with 1, 2, 3 or 4 substituents. Fluorene repeat units may be 2,7-linked fluorene repeat units.

Fluorene repeat units preferably have two substituents in the 9-position thereof. Aromatic carbon atoms of fluorene repeat units may each independently be unsubstituted or substituted with a substituent.

If present, an electron transporting layer located between the light-emitting layers and cathode preferably has a LUMO level of around 1.8-2.7 eV as measured by cyclic voltammetry. An electron-transporting layer may have a thickness in the range of about 5-50 nm

A charge-transporting layer or charge-blocking layer may be crosslinked, particularly if a layer overlying that charge-transporting or charge-blocking layer is deposited from a solution. The crosslinkable group used for this crosslinking may be a crosslinkable group comprising a reactive double bond such and a vinyl or acrylate group, or a benzocyclobutane group. The crosslinkable group may be provided as a substituent of, or may be mixed with, a charge-transporting or charge-blocking material used to form the charge-transporting or charge-blocking layer.

A charge-transporting layer adjacent to a light-emitting layer containing a phosphorescent light-emitting material preferably contains a charge-transporting material having a lowest triplet excited state (T₁) excited state that is no more than 0.1 eV lower than, preferably the same as or higher than, the T₁ excited state energy level of the phosphorescent light-emitting material(s) in order to avoid quenching of triplet excitons.

A charge-transporting layer as described herein may be non-emissive, or may contain a light-emitting material such that the layer is a charge transporting light-emitting layer. If the charge-transporting layer is a polymer then a light-emitting dopant may be provided as a side-group of the polymer, a repeat unit in a backbone of the polymer, or an end group of the polymer. Optionally, a hole-transporting polymer as described herein comprises a phosphorescent polymer in a side-group of the polymer, in a repeat unit in a backbone of the polymer, or as an end group of the polymer.

The polystyrene-equivalent number-average molecular weight (Mn) measured by gel permeation chromatography of the polymers described herein may be in the range of about 1×10³ to 1×10⁸, and preferably 1×10⁴ to 5×10⁶. The polystyrene-equivalent weight-average molecular weight (Mw) of the polymers described herein may be 1×10³ to 1×10⁸, and preferably 1×10⁴ to 1×10⁷.

Polymers as described herein are suitably amorphous.

Hole Injection Layers

A conductive hole injection layer, which may be formed from a conductive organic or inorganic material, may be provided between the anode 101 and the light-emitting layer 103 of an OLED as illustrated in FIG. 1 to assist hole injection from the anode into the layer or layers of semiconducting polymer. Examples of doped organic hole injection materials include optionally substituted, doped poly(ethylene dioxythiophene) (PEDOT), in particular PEDOT doped with a charge-balancing polyacid such as polystyrene sulfonate (PSS) as disclosed in EP 0901176 and EP 0947123, polyacrylic acid or a fluorinated sulfonic acid, for example Nafion®; polyaniline as disclosed in U.S. Pat. No. 5,723,873 and U.S. Pat. No. 5,798,170; and optionally substituted polythiophene or poly(thienothiophene). Examples of conductive inorganic materials include transition metal oxides such as VOx, MoOx and RuOx as disclosed in Journal of Physics D: Applied Physics (1996), 29(11), 2750-2753.

Cathode

The cathode 105 is selected from materials that have a workfunction allowing injection of electrons into the light-emitting layer of the OLED. Other factors influence the selection of the cathode such as the possibility of adverse interactions between the cathode and the light-emitting material. The cathode may consist of a single material such as a layer of aluminium. Alternatively, it may comprise a plurality of conductive materials such as metals, for example a bilayer of a low workfunction material and a high workfunction material such as calcium and aluminium, for example as disclosed in WO 98/10621. The cathode may comprise elemental barium, for example as disclosed in WO 98/57381, Appl. Phys. Lett. 2002, 81(4), 634 and WO 02/84759. The cathode may comprise a thin (e.g. 1-5 nm) layer of metal compound, in particular an oxide or fluoride of an alkali or alkali earth metal, between the organic layers of the device and one or more conductive cathode layers to assist electron injection, for example lithium fluoride as disclosed in WO 00/48258; barium fluoride as disclosed in Appl. Phys. Lett. 2001, 79(5), 2001; and barium oxide. In order to provide efficient injection of electrons into the device, the cathode preferably has a workfunction of less than 3.5 eV, more preferably less than 3.2 eV, most preferably less than 3 eV. Work functions of metals can be found in, for example, Michaelson, J. Appl. Phys. 48(11), 4729, 1977.

The cathode may be opaque or transparent. Transparent cathodes are particularly advantageous for active matrix devices because emission through a transparent anode in such devices is at least partially blocked by drive circuitry located underneath the emissive pixels. A transparent cathode comprises a layer of an electron injecting material that is sufficiently thin to be transparent. Typically, the lateral conductivity of this layer will be low as a result of its thinness. In this case, the layer of electron injecting material is used in combination with a thicker layer of transparent conducting material such as indium tin oxide.

It will be appreciated that a transparent cathode device need not have a transparent anode (unless, of course, a fully transparent device is desired), and so the transparent anode used for bottom-emitting devices may be replaced or supplemented with a layer of reflective material such as a layer of aluminium. Examples of transparent cathode devices are disclosed in, for example, GB 2348316.

Encapsulation

Organic optoelectronic devices tend to be sensitive to moisture and oxygen. Accordingly, the substrate preferably has good barrier properties for prevention of ingress of moisture and oxygen into the device. The substrate is commonly glass, however alternative substrates may be used, in particular where flexibility of the device is desirable. For example, the substrate may comprise one or more plastic layers, for example a substrate of alternating plastic and dielectric barrier layers or a laminate of thin glass and plastic.

The device may be encapsulated with an encapsulant (not shown) to prevent ingress of moisture and oxygen. Suitable encapsulants include a sheet of glass, films having suitable barrier properties such as silicon dioxide, silicon monoxide, silicon nitride or alternating stacks of polymer and dielectric or an airtight container. In the case of a transparent cathode device, a transparent encapsulating layer such as silicon monoxide or silicon dioxide may be deposited to micron levels of thickness, although in one preferred embodiment the thickness of such a layer is in the range of 20-300 nm. A getter material for absorption of any atmospheric moisture and/or oxygen that may permeate through the substrate or encapsulant may be disposed between the substrate and the encapsulant.

Formulation Processing

A formulation suitable for forming a charge-transporting or light-emitting layer may be formed from a compound of formula (I), any further components of the layer such as light-emitting dopants, and one or more suitable solvents.

The formulation may be a solution of the compound of formula (I) and any other components in the one or more solvents, or may be a dispersion in the one or more solvents in which one or more components are not dissolved. Preferably, the formulation is a solution.

Solvents suitable for dissolving compounds of formula (I) are solvents comprising alkyl substituents for example benzenes substituted with one or more C₁₋₁₀ alkyl or C₁₋₁₀ alkoxy groups, for example toluene, xylenes and methylanisoles.

Particularly preferred solution deposition techniques including printing and coating techniques such spin-coating, inkjet printing and slot-die coating.

Spin-coating is particularly suitable for devices wherein patterning of the light-emitting layer is unnecessary—for example for lighting applications or simple monochrome segmented displays.

Inkjet printing is particularly suitable for high information content displays, in particular full colour displays. A device may be inkjet printed by providing a patterned layer over the first electrode and defining wells for printing of one colour (in the case of a monochrome device) or multiple colours (in the case of a multicolour, in particular full colour device). The patterned layer is typically a layer of photoresist that is patterned to define wells as described in, for example, EP 0880303.

As an alternative to wells, the ink may be printed into channels defined within a patterned layer. In particular, the photoresist may be patterned to form channels which, unlike wells, extend over a plurality of pixels and which may be closed or open at the channel ends.

Other solution deposition techniques include dip-coating, roll printing and screen printing.

### EXAMPLES

### Compound Example 1

Compound Example 1 was prepared according to the following reaction scheme:

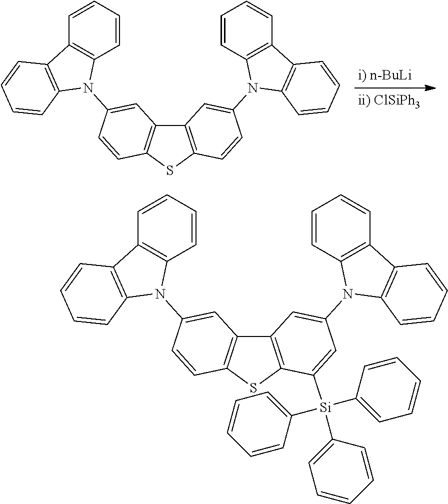

Dicarbazolyl-dibenzothiophene (36 g, 70 mmol) was dissolved in anhydrous THF (500 mL) in a dry, nitrogen-purged flask. The stirred solution was cooled to ˜−50° C. using a CO₂/MeCN cooling bath and n-butyllithium (28 mL, 70 mmol, 2.5 M) was added dropwise over ˜15 mins causing the reaction mixture to turn a deep red colour. The stirred mixture was allowed to warm to ˜−15° C. and stirred for 1.5 h at this temperature. The reaction was re-cooled to −78° C. using a CO₂/acetone cooling bath and a solution of chlorotriphenylsilane (24.75 g, 74 mmol) in anhydrous THF (50 mL) was added dropwise to give a pale red solution. The mixture was allowed to warm to room temperature overnight and quenched by addition of water. The mixture was transferred to a separating funnel and the mixtures were separated. The organics were washed with brine, dried with MgSO4, filtered and concentrated to give a white foamy solid. This crude material was purified by column chromatography on silica eluting with a heptanes/DCM mixture. Product-containing fractions were combined and concentrated to give a white solid which was recrystallised 5 times from a toluene-acetonitrile mixture to yield the product as a white powder (25.6 g, 47%). HPLC indicated a purity of 99.86%.

¹H NMR (THF-d₈) δ [ppm] −8.75 (s, 1H), 8.64 (s, 1H), 8.14 (d, 2H), 8.08 (d, 2H), 8.05 (d, 2H), 7.83 (s, 1H), 7.77 (d, 6H), 7.67 (d, 1H), 7.46 (t, 3H), 7.39-7.46 (m, 10H), 7.34 (t, 2H), 7.30 (t, 2H), 7.22 (t, 2H), 7.18 (t, 2H).

### Compound Example 2

Compound Example 1 was prepared according to the following reaction scheme:

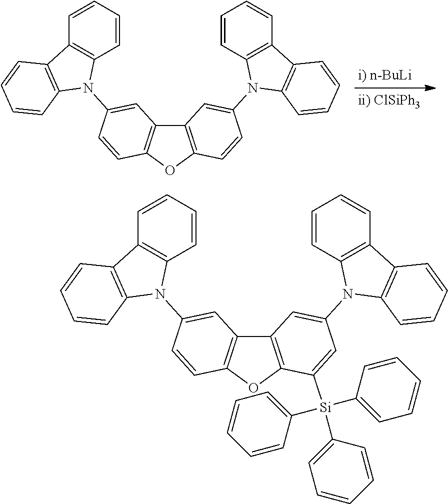

Dicarbazolyl-dibenzothiophene (12 g, 24 mmol) was dissolved in anhydrous THF (600 mL) in a dry, nitrogen-purged flask. The stirred solution was cooled to ˜−40° C. using a CO₂/MeCN cooling bath and n-butyllithium (15 mL, 24 mmol, 1.6 M) was added dropwise over ˜60 mins. The stirred mixture was warmed to 50° C. for 18 h. The reaction was re-cooled to −78° C. using a CO₂/acetone cooling bath and a solution of chlorotriphenylsilane (7 g, 24 mmol) was slowly added. The mixture was allowed to warm to room temperature overnight and quenched by addition of 1.5 N HCl. The mixture was transferred to a separating funnel and the mixtures were separated. The aqueous layer was extracted with ethyl acetate (500 mL) and the organics were washed with water, dried with MgSO4, filtered and concentrated. The residue was purified by column chromatography on silica eluting with a hexanes/ethyl acetate mixture. Product-containing fractions were combined and concentrated to give a white solid which was purified further by column chromatography on reverse-phase silica eluting with an acetonitrile/THF mixture. Product-containing fractions were concentrated to give white powder (9.7 g, 53%). HPLC indicated a purity of 99.70%.

¹H NMR (DMSO-d₆) δ [ppm] −8.79 (d, 1H), 8.64 (d, 1H), 8.26 (d, 2H), 7.85 (d, 1H), 7.68-7.73 (m, 7H), 7.39-7.51 (m, 18H), 7.26-7.31 (m, 4H).

HOMO and LUMO levels of Compound Example 1 and of Comparative Example 1 are given in Table 1.

TABLE 1

| | Measured values (eV) | | Modelled values (eV) | |
|---|---|---|---|---|
| Compound | LUMO | HOMO | LUMO | HOMO |
| Comparative | −1.96 | −5.73 | −1.433 | −5.356 |
| Compound 1 | | | | |
| Compound Example 1 | −2.21 | −5.76 | −1.376 | −5.28 |
| Compound Example 2 | −2.12 | −5.68 | −1.436 | −5.309 |

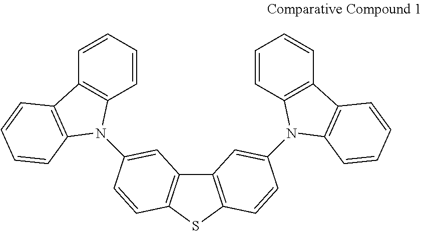 [SMILES: c1ccc2c(c1)c1ccccc1n2-c1ccc2sc3ccc(-n4c5ccccc5c5ccccc54)cc3c2c1]

Computer modelling of HOMO and LUMO levels was performed using Gaussian09 RevC.01. The modelled LUMO levels of Comparative Compound 1 and Compound Example 1 are similar, with the LUMO of Compound Example 1 being slightly shallower. This is expected because the triphenylsilyl group is not expected to inductively withdraw or donate electrons and there is no conjugation between the silicon atom and the dibenzothiophene it is bound to. It is therefore surprising that the LUMO level of Compound Example 1 is considerably deeper (further from vacuum) than that of Comparative Compound 1.

### Composition Examples

The photoluminescent quantum yield (PLQY) of a composition of Compound Example 1 with Blue Phosphorescent Emitter 1 was prepared. The composition has a PLQY value of about 50%. In comparison, the PLQY of a composition of Comparative Compound 1 with Blue Phosphorescent Emitter 1 was about 60%.

Blue Phosphorescent Emitter 1 has the following structure:

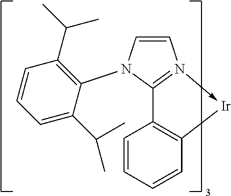

### White Device Example 1

A white organic light-emitting device having the following structure was prepared:

ITO/HIL/LEL (R)/LEL (G, B)/ETL/Cathode

wherein ITO is an indium-tin oxide anode; HIL is a hole-injecting layer comprising a hole-injecting material, LEL (R) a hole-transporting, red light-emitting layer, LEL (G, B) is a light-emitting layer containing Compound Example 1 and a blue and green phosphorescent material, and ETL is an electron-transporting layer.

A substrate carrying ITO was cleaned using UV/Ozone. A hole injection layer was formed to a thickness of about 65 nm by spin-coating a formulation of a hole-injection material. A red light-emitting hole transporting layer was formed to a thickness of about 17 nm by spin-coating a crosslinkable red light-emitting hole-transporting polymer and crosslinking the polymer by heating at 180° C. The green and blue light-emitting layer was formed to a thickness of about 80 nm by spin-coating Compound Example 1 (74 wt %), Blue Phosphorescent Emitter 1 (25 wt %) and Green Phosphorescent Emitter 1 (1 wt %). An electron-transporting layer was formed on the light-emitting layer from a polymer as described in WO 2012/133229. A cathode was formed on the electron-transporting layer of a first layer of sodium fluoride of about 2 nm thickness, a layer of silver of about 100 nm thickness and a layer of aluminium of about 100 nm thickness.

Green Phosphorescent Emitter 1 is a tris(phenylpyridine)iridium wherein each ligand is substituted with an alkylated dendron of formula (XIb).

The hole-transporting layer was formed by spin-coating a polymer comprising 1,4-phenylene repeat units substituted with crosslinkable groups; amine repeat units of formula (VII-1); and a red phosphorescent emitting repeat unit formed from the following monomer:

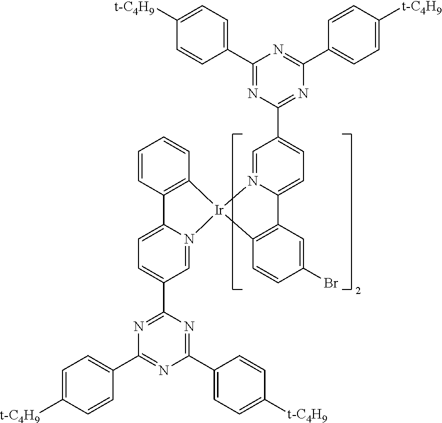

The electron-transporting layer was formed by spin-coating a polymer comprising the cesium salt of electron-transporting unit 1 as described in WO 2012/133229 to a thickness of 10 nm.

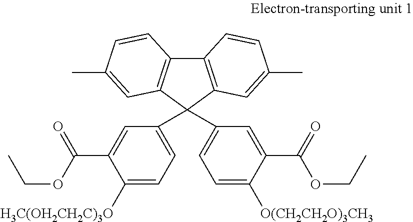

Comparative White Device 1

A device was prepared as described for White Device Example 1 except that Comparative Compound 1 was used in place of Compound Example 1.

Results are given in Table 2.

The PLQY measurements suggest that Comparative Device 1, containing Comparative Compound 1, will give a higher device efficiency. Surprisingly, however, Device Example 1 has a higher efficiency and requires a lower drive voltage to reach 1000 cd/m² or a current density of 10 mA/cm² than Comparative Device 1.

TABLE 2

| | Voltage | Voltage | Efficiency | Efficiency | External Quantum | |
|---|---|---|---|---|---|---|
| | at 1,000 | at 10 | at 1,000 | at 1,000 | Efficiency | |
| | cd/m² | mA/cm² | cd/m² | cd/m² | at 1,000 cd/m² | Max |
| | brightness | current density | brightness | brightness | brightness | EQE |
| Device | (V) | (V) | (Lm/W) | (Cd/A) | (%) | (%) |
| Comparative | 4.19 | 5.15 | 28.99 | 38.81 | 15.78 | 16.55 |
| Device 1 | | | | | | |
| Device | 3.94 | 4.95 | 34.59 | 43.34 | 17.90 | 18.73 |
| Example 1 | | | | | | |

### White Device Example 2

A device was prepared as described for Device Example 1 except that Blue Phosphorescent Emitter 2 (30 mol %) was used in place of Blue Phosphorescent Emitter 1.

Blue Phosphorescent Emitter 2 has the following structure:

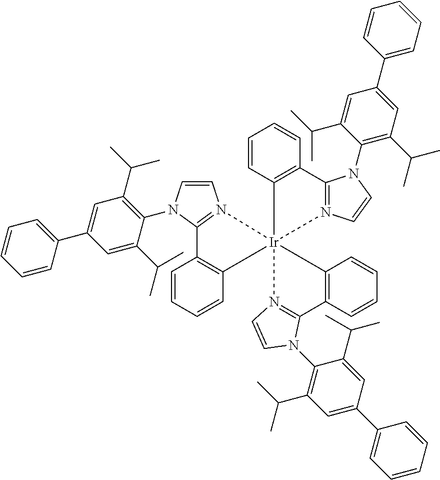

### White Device Example 3

A device was prepared as described for Device Example 2 except that 22 mol % of Blue Phosphorescent Emitter 2 was used.

Comparative White Device 2

A device was prepared as described for White Device Example 2 except that Comparative Compound 1 was used in place of Compound Example 1 with 13.5 mol % of Blue Phosphorescent Emitter 2.

Results are provided in Table 3. Device Example 2 has a higher efficiency and requires a lower drive voltage to reach 1000 cd/m² or a current density of 10 mA/cm² than Comparative Device 2.

TABLE 3

| | V | V | Lm/W | Cd/A | EQE | |
|---|---|---|---|---|---|---|
| | at 1 | at 10 | at 1 | at 1 | at 1 | Max |
| Device | kcd/m² | mA/cm² | kcd/m² | kcd/m² | kcd/m² | EQE |
| Comparative | 3.77 | 4.64 | 37.5 | 44.9 | 17.3 | 18.2 |
| Device 2 | | | | | | |
| Device | 3.65 | 4.60 | 41.3 | 48.1 | 18.7 | 19.4 |
| Example 2 | | | | | | |
| Device | 3.60 | 4.43 | 37.9 | 43.5 | 18.4 | 19.7 |
| Example 3 | | | | | | |

### White Device Example 4

A white organic light-emitting device having the following structure was prepared:

ITO/HIL/LEL (R)/LEL (G, B)/ETL/Cathode

wherein ITO is an indium-tin oxide anode; HIL is a hole-injecting layer comprising a hole-injecting material, LEL (R) a hole-transporting, red light-emitting layer, LEL (G, B) is a light-emitting layer containing Compound Example 1 and a blue and green phosphorescent material, and ETL is an electron-transporting layer.

A substrate carrying ITO (45 nm) was cleaned using UV/Ozone. A hole injection layer was formed to a thickness of about 65 nm by spin-coating a formulation of a hole-injection material. A red light-emitting hole transporting layer was formed to a thickness of about 17 nm by spin-coating a crosslinkable hole-transporting polymer formed by Suzuki polymerisation and comprising 1,4-phenylene repeat units and repeat units of formula (VII-1) endcapped with Red Phosphorescent End-Capping Group 1 and crosslinking the polymer by heating at 180° C. The green and blue light-emitting layer was formed to a thickness of about 65 nm by spin-coating Compound Example 1 (69 wt %), Blue Phosphorescent Emitter 3 (12 wt %), Hole-transporting complex 1 (18 wt %) and a green phosphorescent emitter of tris(phenylpyridine)iridium emitter wherein each ligand is substituted with an alkylated 3,5-diphenylbenzene dendron (1 wt %). An electron-transporting layer was formed on the light-emitting layer from a polymer as described in WO 2012/133229. A cathode was formed on the electron-transporting layer of a first layer of sodium fluoride of about 3.5 nm thickness, a layer of aluminium of about 100 nm thickness and a layer of silver of about 100 nm thickness.

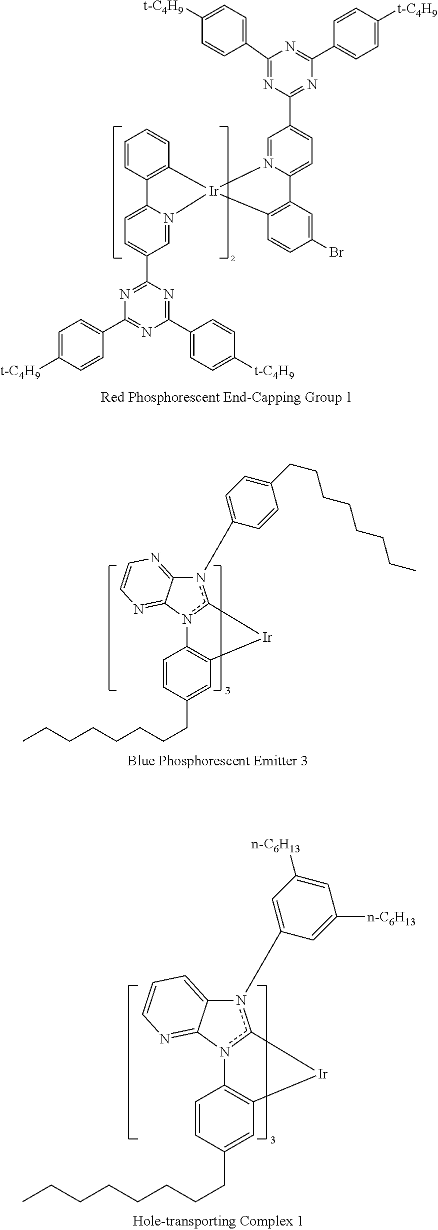

Comparative White Device 4

A device was prepared as described for White Device Example 4 except that Comparative Compound 1 was used in place of Compound Example 1.

Results are given in Table 4.

TABLE 4

| | | | | External | |
|---|---|---|---|---|---|
| | Voltage | Voltage | | Quantum | |
| | at | at 1 | Efficiency | Efficiency | |
| | 1,000 | mA/cm² | at 1,000 | at 1,000 | |
| | cd/m² | current | cd/m² | cd/m² | Max |
| | brightness | density | brightness | brightness | EQE |
| Device | (V) | (V) | (Lm/W) | (%) | (%) |
| White Device | 4.9 | 4.3 | 23.6 | 15.4 | 16.2 |
| Example 4 | | | | | |
| Comparative | 6.4 | 5.6 | 18.8 | 13.6 | 14.1 |
| White Device | | | | | |
| 4 | | | | | |

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A compound of formula (I) 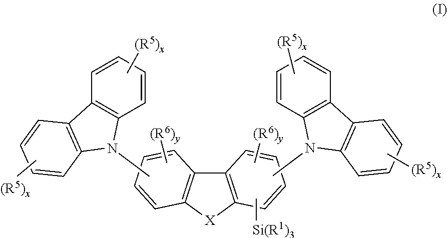
wherein:
X is O, S, NR⁸, CR⁸₂ or SiR⁸₂ wherein R⁸ in each occurrence is independently a substituent;
R¹, R⁵ and R⁶ are independently in each occurrence a substituent;
x independently in each occurrence is 0, 1, 2, 3 or 4; and
y independently in each occurrence is 0, 1 or 2.

2. The compound according to claim 1, wherein each R¹ independently is (Ar¹)ₚ wherein Ar¹ independently in each occurrence is a C₆₋₂₀ aryl or heteroaryl group that may be unsubstituted or substituted with one or more substituents, and p is 1 or more.

3. The compound according to claim 2, wherein each R¹ is phenyl which may independently in each occurrence be unsubstituted or substituted with one or more C₁₋₁₀ alkyl groups.

4. The compound according to claim 1, wherein each x is 0.

5. The compound according to claim 1, wherein each y is 0.

6. The compound according to claim 1, wherein X is S or O.

7. A composition comprising a compound according to claim 1, and at least one light-emitting dopant.

8. The composition according to claim 7, wherein the light-emitting dopant is a phosphorescent dopant.

9. The composition according to claim 7, wherein the light-emitting dopant is a blue light-emitting material.

10. The composition according to claim 7, wherein the light-emitting dopant is a metal complex comprising at least one unsubstituted or substituted phenylimidazole ligand.

11. A formulation comprising a compound according to claim 1, and one or more solvents.

12. An organic light-emitting device comprising an anode, a cathode and a light-emitting layer between the anode and the cathode wherein the light-emitting layer comprises a compound according to claim 1.

13. An organic light-emitting device comprising an anode, a cathode and a light-emitting layer between the anode and the cathode wherein the light-emitting layer comprises a composition according to claim 7.

14. The organic light-emitting device according to claim 12, wherein the device emits white light.

15. The organic light-emitting device according to claim 14, wherein the device comprises at least one further light-emitting layer.

16. A method of forming an organic light-emitting device according to claim 12, comprising the step of forming the light-emitting layer over one of the anode and the cathode and forming the other of the anode and the cathode over the light-emitting layer.

17. The method according to claim 16 wherein the light-emitting layer is formed by depositing a formulation comprising a compound of formula (I) 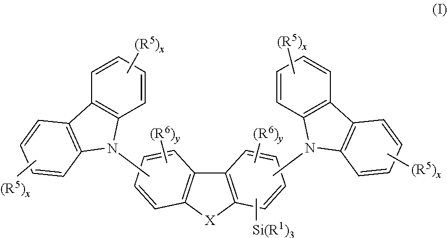
wherein:
X is O, S, NR⁸, CR⁸₂ or SiR⁸₂ wherein R⁸ in each occurrence is independently a substituent;
R¹, R⁵ and R⁶ are independently in each occurrence a substituent;
x independently in each occurrence is 0, 1, 2, 3 or 4;
y independently in each occurrence is 0, 1 or 2;
and one or more solvents and
evaporating the one or more solvents.

18. A polymer having a polymer backbone and comprising a group of formula (II) in the polymer backbone, as a side-chain of the polymer backbone or and end group of the polymer backbone: 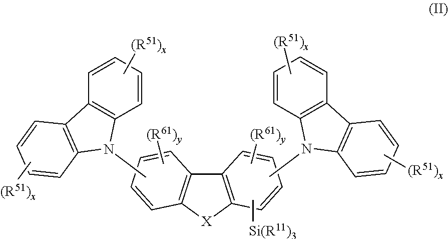
wherein X is O, S, NR⁸, CR⁸₂ or SiR⁸₂ wherein R⁸ in each occurrence is independently a substituent;
R¹¹, R⁵¹ and R⁶¹ are independently in each occurrence a substituent;
x independently in each occurrence is 0, 1, 2, 3 or 4; and
y independently in each occurrence is 0, 1 or 2,
with the proviso that at least one of R⁵¹, R⁶¹ and R¹¹ is bound to the polymer backbone.

19. The polymer according to claim 18, wherein the group of formula (II) is a repeat unit of formula (IIa) or (IIb):

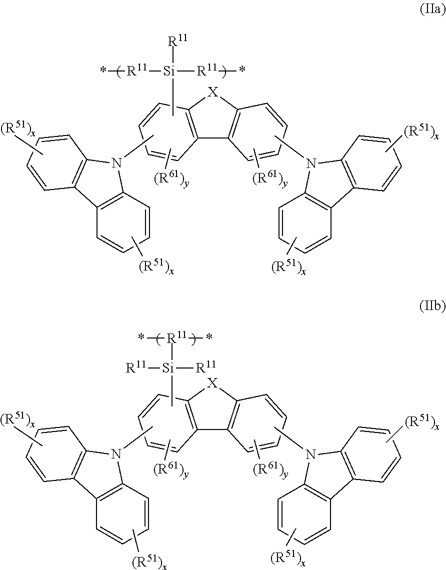

20. A compound of formula (V): 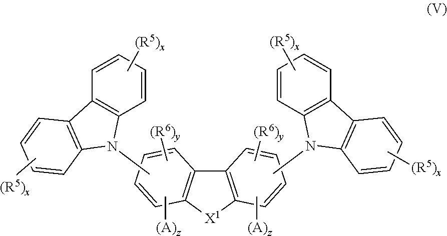
wherein:
X¹ is O or S;
each A is a LUMO-deepening substituent;
R⁵ and R⁶ are independently in each occurrence a substituent;
x independently in each occurrence is 0, 1, 2, 3 or 4;
y independently in each occurrence is 0, 1 or 2, and
each z is independently 0 or 1 with the proviso that at least one z is 1.
